SEQUENCE LISTING (20 sequences)

SEQ ID NO: 1 - DNA, 36 bp, Artificial
  synthetic
gtgcctggcagcaccggagattgtgatattgagggc

SEQ ID NO: 2 - DNA, 39 bp, Artificial
  synthetic
gcctgggcctttggggactcgtgttcctttgtgcccatc

SEQ ID NO: 3 - DNA, 37 bp, Artificial
  synthetic
agagagaccaagacaccagattgtgatattgagggca

SEQ ID NO: 4 - DNA, 47 bp, Artificial
  synthetic
acgggccctctagactcgagtcatcagtgttcctttgtgcccatcag

SEQ ID NO: 5 - DNA, 37 bp, Artificial Sequence
  synthetic
agagagaccaagacaccagatgctcacaagtctgagg

SEQ ID NO: 6 - DNA, 44 bp, Artificial Sequence
  synthetic
acgggccctctagactcgagtcatcacaggcccagggcagcctg

SEQ ID NO: 7 - DNA, 36 bp, Artificial Sequence
  synthetic
gtgcctggcagcaccggagatgctcacaagtctgag

SEQ ID NO: 8 - DNA, 38 bp, Artificial Sequence
  synthetic
gcctgggcctttggggactccaggcccagggcagcctg

SEQ ID NO: 9 - DNA, 27 bp, Artificial Sequence
  synthetic
cgtttaaacttaagcttggtaccgagc

SEQ ID NO: 10 - DNA, 20 bp, Artificial Sequence
  synthetic
tccggtgctgccaggcaccc

SEQ ID NO: 11 - DNA, 20 bp, Artificial Sequence
  synthetic
gagtccccaaaggcccaggc

SEQ ID NO: 12 - DNA, 21 bp, Artificial Sequence
  sythetic
tggtgtcttggtctctctctc

SEQ ID NO: 13 - DNA, 29 bp, Artificial Sequence
  synthetic
cgcgcgacgcgtggctccggtgcccgtca

SEQ ID NO: 14 - DNA, 36 bp, Artificial Sequence
  synthetic
ctactagctagctcacgacacctgaaatggaagaaa

SEQ ID NO: 15 - protein, 152 aa, Artificial Sequence
  synthetic
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 16 - protein, 585 aa, Artificial Sequence [sequence omitted]

SEQ ID NO: 17 - protein, 20 aa, Artificial Sequence
  synthetic
METDTLLLWVLLLWVPGSTG

SEQ ID NO: 18 - protein, 58 aa, Artificial Sequence
  synthetic
ESPKAQASSVPTAQPQAEGSLAKATTAPATTRNTGRGGEEKKKEKEKEEQEERETKTP

SEQ ID NO: 19 - protein, 812 aa, Artificial Sequence
  synthetic
METDTLLLWVLLLWVPGSTGDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHESPKAQASSVPTAQPQAEGSLAKATTAPATTRNTGRGGEEKKKEKEKEEQEERETKTPDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVENLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 20 - protein, 815 aa, Artificial Sequence
  synthetic
METDTLLLWVLLLWVPGSTGDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLESPKAQASSVPTAQPQAEGSLAKATTAPATTRNTGRGGEEKKKEKEKEEQEERETKTPDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

(19) United States
(12) Patent Application Publication
(10) Pub. No.: US 2024/0092851 A1
(43) Pub. Date: Mar. 21, 2024

(54) IL-7 FUSION PROTEIN AND RELATED METHODS

(71) Applicant: JECHO LABORATORIES, INC., Frederick, MD (US)

(72) Inventors: Shusheng Wang, Ellicott City, MD (US); Jianwei Zhu, Frederick, MD (US); Haiqiu Huang, North Bethesda, MD (US); Ailin Wang, Urbana, MD (US); Kaiyong Yang, Potomac, MD (US); Yueqing Xie, Clarksburg, MD (US); Hua Jiang, Clarksburg, MD (US)

(21) Appl. No.: 18/254,223

(22) PCT Filed: Nov. 23, 2021
(86) PCT No.: PCT/US2021/060441

§ 371 Date: May 24, 2023

Related U.S. Application Data
(60) Provisional application No. 63/118,285, filed on Nov. 25, 2020.
(60) Provisional application No. 63/129,817, filed on Dec. 23, 2020.

(51) Int. Cl. C07K 14/54; A61P 37/04; C07K 14/765

(52) U.S. Cl. CPC C07K 14/5418; A61P 37/04; C07K 14/765; A61K 38/00; C07K 2319/31

(57) ABSTRACT

The disclosure provides IL-7 fusion proteins as well as compositions comprising them. The disclosure further provides methods of treating and/or preventing lymphopenia or immunodeficiency in a subject, wherein the method includes administering a fusion protein as described herein.

### CROSS REFERENCE TO RELATED APPLICATIONS

This application claims priority to U.S. Provisional Patent Application Ser. No. 63/118,285, filed Nov. 25, 2020, and to U.S. Provisional Patent Application Ser. No. 63/129,817, filed Dec. 23, 2020, both of which are incorporated herein by reference in their entirety.

### INCORPORATION BY REFERENCE

This application incorporates by reference a Sequence Listing submitted with this application in computer readable form (CRF) as a text file entitled “64647WO01_SeqList”.

### BACKGROUND

Interleukin-7 (IL-7) is an immune modulator produced by stromal cells in lymphoid tissue. IL-7 is involved in lymphocyte homeostasis, specifically for the development of T cells and their maintenance and preservation in the periphery via interactions with its receptor, interleukin-7 receptor (IL-7R), which is expressed by cells of the lymphoid lineage. In the thymus, IL-7 directly stimulates recombination of the T-cell receptor γ-chain (TCRγ) locus to increase the antigen diversity of T cells. In circulating T cells, IL-7 induces expression or activity of anti-apoptotic factors and proliferation signals, as well as reduces apoptotic factors, resulting in T cell proliferation.

Its immunostimulatory effect makes IL-7 an attractive candidate therapeutic for immunodeficiency treatment and prevention, however, there is a need for strategies to extend the serum half-life of IL-7.

Among other aspects, described herein are novel IL-7 fusion proteins that extend the half-life of the IL-7 protein, and can be used in methods for treatment and prevention of immunodeficiency.

### SUMMARY OF THE DISCLOSURE

The present disclosure generally provides an interleukin 7 (IL-7) fusion protein. In one aspect, the disclosure provides an IL-7 fusion protein that binds IL-7 receptor.

In some embodiments, a fusion protein comprises an IL-7 protein, an albumin protein, and a hinge region that joins the IL-7 protein and the albumin protein. In some aspects, the IL-7 protein is human IL-7 and the albumin protein is human serum albumin. In some embodiments, the human IL-7 comprises the amino acid sequence of SEQ ID NO: 15. In some embodiments, the human serum albumin (HSA) comprises the amino acid sequence of SEQ ID NO: 16. In some embodiments, the hinge region is human IgD. In some embodiments, the hinge region comprises the amino acid sequence of SEQ ID NO: 18. The IL-7 protein can be disposed toward the C-terminus of the fusion protein and the HSA disposed toward the N-terminus of the fusion protein. Alternatively, the HSA protein can be disposed toward the C-terminus of the fusion protein and the IL-7 protein disposed toward the N-terminus of the fusion protein.

In some embodiments, the fusion protein further comprises a signal peptide. The signal peptide can be a mouse IgG peptide. In some embodiments, the signal peptide comprises the amino acid sequence of SEQ ID NO: 17.

In some embodiments, the fusion protein comprises an amino acid sequence of SEQ ID NO: 19. On other embodiments, the fusion protein comprises an amino acid sequence of SEQ ID NO: 20.

The fusion proteins disclosed herein can extend the serum half-life of the IL-7 protein relative to recombinant or exogenous human IL-7 when administered to a subject. Various strategies can be used to extend protein serum half-life. For example, the fusion protein described here Human Serum Albumin (HSA)-IL7 comprises one, two, or three or more features. One feature comprises HSA as a fusion domain or fusion partner. HSA is the most abundant serum protein and has a serum-half life that is equivalent or better than IgG. Another feature comprises an IgD hinge region as the linker between domains (IL-7 and HSA). The IgD hinge is the longest hinge region among all immunoglobin and confers flexibility, allowing IL-7 to maintain its activity in the fusion format. A third feature is that IL-7 is linked to the C terminus of the HSA. This format provides the better biochemical and biophysical properties when compared to an N terminal location for the fusion to IL-7.

In any of the aspects and embodiments described herein, the IL-7 fusion proteins disclosed herein can be used to treat and/or prevent lymphopenia in a subject. In some aspects, the IL-7 fusion proteins can stimulate white blood cell proliferation, lymphocyte proliferation, and/or lymphocyte differentiation in a subject. In other aspects, the IL-7 fusion proteins can treat and/or prevent immunodeficiency in a subject. Subjects that can benefit from the disclosed IL-7 fusion proteins include, without limitation, those having cancer, human immunodeficiency virus, hepatitis B, hepatitis C, lymphopenia, sepsis, and subjects having undergone stem cell, tissue, or organ transplantation. In certain aspects, the IL-7 fusion protein can increase white blood cells counts, increase lymphocyte counts, and/or increase T cell subpopulations, such as CD3⁺CD4⁺ T cells, CD3+CD4⁺ T cells, and CD3⁺ T cells in a subject. The methods disclosed herein comprise administering an IL-7 fusion protein to a subject.

Additional aspects of the disclosure will be apparent to one of ordinary skill in the art in view of the following description and illustrative examples.

### BRIEF DESCRIPTION OF THE DRAWINGS

FIG. 1A illustrates an embodiment of an IL-7 fusion protein.

FIG. 1B illustrates another embodiment of an IL-7 fusion protein.

FIG. 2 illustrates the pharmacokinetic profiles of JL18008-1 (IL-7-HSA) and JL18008-2 (HSA-IL-7) fusion proteins.

FIGS. 3A-3E illustrate recovery of immune cell populations following in vivo administration of JL18008-2 to lymphopenic mice.

FIG. 4 illustrates the in vitro bioactivity of JL18008-2.

### DETAILED DESCRIPTION

Before continuing to describe the present disclosure in further detail, it is to be understood that this disclosure is not limited to specific proteins, nucleic acids, compositions, or process steps, as such can vary while still falling within the scope of the description provided herein. It must be noted that, as used in this specification and the appended claims, the singular form “a”, “an” and “the” include plural referents unless the context clearly dictates otherwise.

Unless defined otherwise, all technical and scientific terms used herein have the same meaning as commonly understood by one of ordinary skill in the art to which this invention is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this invention.

Amino acids can be referred to herein by either their commonly known three letter symbols or by the one-letter symbols recommended by the IUPAC-IUB Biochemical Nomenclature Commission. Nucleotides, likewise, can be referred to by their commonly accepted single-letter codes.

### A. Fusion Proteins

As used herein, the term “fusion protein” refers to a polypeptide construct generated through the joining and expression of two or more genes encoding distinct polypeptides. The translation of the fusion gene results in a single polypeptide with functional properties derived from each of the original polypeptides. The polypeptides can be fused directly or joined via a linker or hinge.

Described herein are novel IL-7 fusion proteins. As used herein, the term “interleukin-7” or “IL-7” refers to IL-7 polypeptides or derivatives thereof having substantial amino acid sequence identify to a mature, wild-type mammalian IL-7 For example, IL-7 can refer to an amino acid sequence of a recombinant or non-recombinant polypeptide having an amino acid sequence of: i) a native allelic variant of an IL-7 polypeptide, ii) a biologically active fragment of an IL-7 polypeptide, or iii) a biologically active variant or analog of an IL-7 polypeptide. The IL-7 of the fusion protein can be obtained from any mammalian species, or obtained from a recombinant expression system (e.g., yeast, bacteria, etc.). The IL-7 may be glycosylated, partially glycosylated, or non-glycosylated.

In one aspect, an IL-7 fusion protein comprises IL-7, a hinge region, and an albumin protein, such as but not limited to, human serum albumin (HSA). In some embodiments, the IL-7 fusion proteins comprise less than the full-translated sequence of HSA. The IL-7 fusion protein can further comprise a signal peptide at the N-terminus of the fusion protein, which may be processed (e.g., cleaved) when the fusion protein is expressed or produced in certain host cells (e.g., mammalian host cells such as 293F or CHO-K1). In some embodiments, the IL-7 polypeptide of the fusion protein is human IL-7.

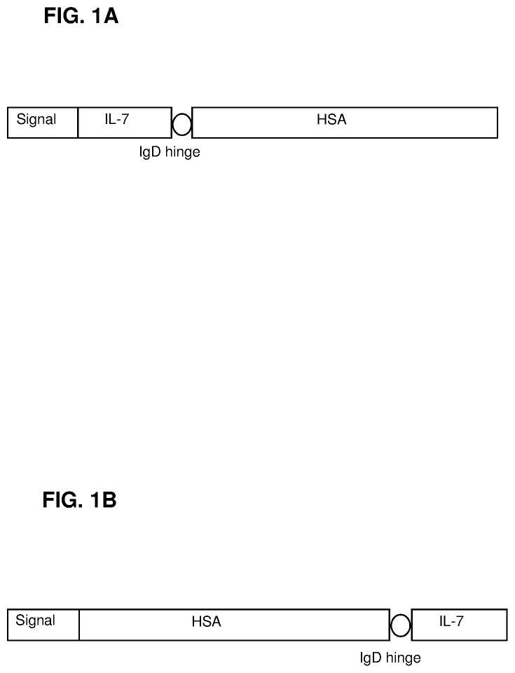

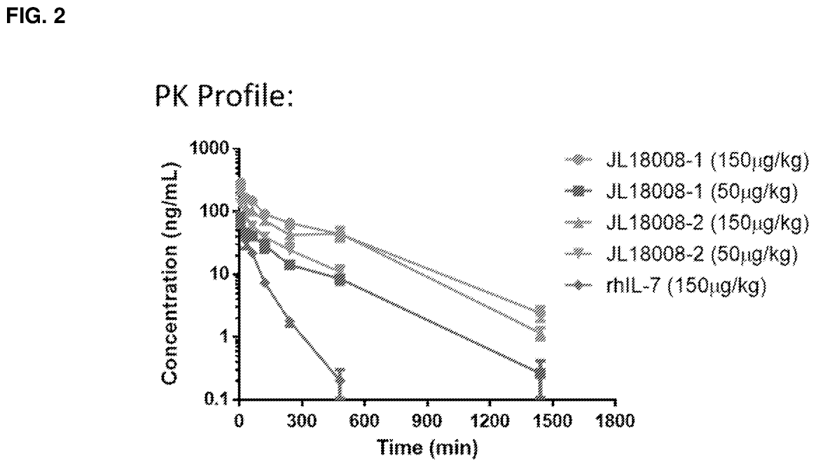

In one aspect, the IL-7 is disposed toward the N-terminus of the fusion protein and the HSA is disposed toward the C-terminus, with the hinge region disposed there between (FIG. 1A). In another aspect, the IL-7 is disposed toward the C-terminus of the fusion protein and the HSA is disposed toward the N-terminus with the hinge region disposed there between (FIG. 1B).

As used herein, the term “human serum albumin” refers to a HSA polypeptide or derivative thereof having substantial amino acid sequence identify to mature, wild-type human HSA, which is can be used as carrier protein at least because of its long serum half-life of up to 21 days. Other carrier proteins, aside from HSA, are also contemplated. In some aspects, the carrier protein is a heavy chain immunoglobulin constant domain derived from IgA, IgD, IgE, IgG, or IgM. In other aspects and embodiments, IL-7 can be modified by pegylation.

Fusion proteins of the disclosure are joined by the fusion of the C-terminal region (e.g., C-terminus) of one polypeptide or protein (IL-7 or HSA) to the N-terminal region (e.g., N-terminus) of a hinge or linker peptide and the fusion of the N-terminal region (e.g., N-terminus) of a second polypeptide or protein (IL-7 or HSA) to the C-terminal region (e.g., C-terminus) of the 1^{st} protein-hinge or linker complex. The hinge or linker peptide comprises an amino acid sequence that provides a covalent linkage between the protein domains (IL-7 and HSA), and can be immediately C-terminal to the last amino acid of the IL-7 or HSA. The hinge or linker region can be any peptide linker that is generally known and used in the art to generate fusion proteins. In some embodiments the hinge or linker is derived from the hinge region of an immunoglobulin such as, for example, an IgA, IgD, IgE, IgG, or IgM amino acid sequence. As used herein, “hinge region” refers to a polypeptide comprising an amino acid sequence that shares sequence identify or similarity with a portion of an immunoglobulin hinge region sequence. Accordingly, a “hinge region” encompasses fragments of the immunoglobulin hinge region that allow the linked polypeptides to achieve a biologically active conformation. Hinge regions in some example embodiments of the present disclosure share at least 70-80% sequence identify to a wild-type immunoglobulin hinge region amino acid sequence, preferably, greater than about 90% sequence identify. In some particular aspects, the hinge region comprises a hinge region from, or derived from, human IgD or IgG. Human IgD, which lacks cysteine and is the longest immunoglobulin hinge, provides increased fusion protein flexibility. Such flexibility can increase engagement or binding of the IL-7 fusion protein with the IL-7 receptor.

As used herein, “signal peptide” refers to a 5-30 amino acid peptide present at the N-terminus of the fusion protein. In some aspects, the fusion protein comprises an N-terminal signal peptide derived from an immunoglobulin amino acid sequence. In certain aspects, the signal peptide can be a mouse IgG peptide, specifically a mouse IgG kappa light chain, which lends to efficient expression and secretion of the fusion protein.

In some aspects, the fusion proteins disclosed herein can be characterized by one or more of the following structural and/or functional properties:
a. an amino acid sequence comprising: (i) human IL-7 (hIL-7) of SEQ ID NO: 15, (ii) human serum albumin (HSA) of SEQ ID NO: 16, (iii) a signal peptide of SEQ ID NO: 17, and (iv) a hinge peptide of SEQ ID NO: 18;
b. an amino acid sequence comprising: (i) human IL-7 (hIL-7) of SEQ ID NO: 15, (ii) human serum albumin (HSA) of SEQ ID NO: 16, and (iii) a hinge peptide of SEQ ID NO: 18;
c. binding specificity for IL-7 receptor;
d. an extended half-life relative to recombinant IL-7 in human serum;
e. an EC50 having a potency within 5-fold of recombinant IL-7 (e.g., 5× more or less potent);
f. the ability to extend the serum half-life of the IL-7 protein relative to recombinant or exogenous IL-7 proteins when administered to a subject;
g. the ability to stimulate white blood cell proliferation, lymphocyte proliferation, and/or lymphocyte differentiation; and
h. the ability to treat, attenuate, or reduce in severity lymphopenia or immunodeficiency in a subject.

In some aspects, an IL-7 fusion protein comprises an IL-7 protein having SEQ ID NO: 15.

In some aspects, an IL-7 fusion protein comprises HSA having SEQ ID NO: 16.

In some aspects, an IL-7 fusion protein comprises a hinge region having SEQ ID NO: 18.

In some aspects, an IL-7 fusion protein comprises a signal peptide having SEQ ID NO: 17.

In some aspects, an IL-7 fusion protein comprises human IL-7 (hIL-7) of SEQ ID NO: 15, HSA of SEQ ID NO: 16, a signal peptide of SEQ ID NO: 17, and a hinge peptide of SEQ ID NO: 18.

In some aspects, an IL-7 fusion protein comprises human IL-7 (hIL-7) of SEQ ID NO: 15, HSA of SEQ ID NO: 16, and a hinge peptide of SEQ ID NO: 18.

In some aspects, an IL-7 fusion protein comprises SEQ ID NO: 19.

In some aspects, an IL-7 fusion protein comprises SEQ ID NO: 20.

In still further aspects, the disclosure provides JL18008-1 and JL18008-2.

In some aspects, a fusion protein comprises an IL-7 protein having SEQ ID NO: 15.

In some aspects, a fusion protein comprises HSA having SEQ ID NO: 16.

In some aspects, a fusion protein comprises a hinge region having SEQ ID NO: 18.

In some aspects, a fusion protein comprises a signal peptide having SEQ ID NO: 17.

In some aspects, a fusion protein comprises human IL-7 (hIL-7) of SEQ ID NO: 15, HSA of SEQ ID NO: 16, a signal peptide of SEQ ID NO: 17, and a hinge peptide of SEQ ID NO: 18.

In some aspects, a fusion protein comprises human IL-7 (hIL-7) of SEQ ID NO: 15, HSA of SEQ ID NO: 16, and a hinge peptide of SEQ ID NO: 18.

In some aspects, a fusion protein comprises SEQ ID NO: 19.

In some aspects, a fusion protein comprises SEQ ID NO: 20.

As one of ordinary skill can appreciate, the sequences disclosed herein can be modified to some degree without compromising the ability of the fusion protein to interact with a target cell receptor, i.e., IL-7 receptor. In some aspects, fusion protein sequence variants retain the ability to stimulate white blood cell proliferation, lymphocyte proliferation, and/or lymphocyte differentiation in vivo and/or in vitro. In some aspects, fusion protein sequence variants retain the ability to treat, attenuate, or reduce in severity lymphopenia or immunodeficiency in a subject.

As used herein, sequence “variants” refer to a fusion protein amino acid sequence comprising at least one amino acid insertion, deletion, and/or substitution, wherein the resulting fusion protein maintains one or more of its functional characteristics as described herein. An amino acid insertion variant is characterized by the insertion of one or more amino acids between two existing amino acids. An amino acid deletion variant is characterized by the deletion of one or more amino acids from the fusion protein sequence. An amino acid substitution is characterized by at least one amino acid in the sequence being replaced by another amino acid. In embodiments relating to substitutions, the amino acid substitution(s) may be a conservative substitution, (i.e. an amino acid from one family of amino acids (acidic, basic, non-polar, and uncharged, based on side chain characteristics, including size) is substituted with an amino acid from the same family).

In some aspects, the sequence identity between the variant fusion protein sequence and the fusion protein sequences disclosed herein will be at least 85%, 86%, 87%, 88%, 89%, 90%, 91%, 92%, 93%, 94%, 95%, 96%, 97%, 98%, or 99%. “Sequence identity” refers to the percentage of amino acid residues that are identical to the sequences being compared.

### B. Labels, Conjugates, and Moieties

The fusion proteins disclosed herein can be conjugated to a therapeutic agent, solid support, affinity agent, or a detectable agent. Fusion proteins of the disclosure can be conjugated to labels wherein the fusion protein and/or its associated targets(s) can be detected. Labels include, without limitation, a chromophore, a fluorophore, a fluorescent protein, a phosphorescent dye, a tandem dye, a particle, a hapten, an enzyme and a radioisotope.

In certain aspects, the fusion proteins are conjugated to a fluorophore. The choice of the fluorophore attached to the fusion protein will determine the absorption and fluorescence emission properties of the conjugated fusion protein. Physical properties of a fluorophore label can include, but are not limited to, spectral characteristics (absorption, emission and stokes shift), fluorescence intensity, lifetime, polarization and photo-bleaching rate, or combination thereof. All of these physical properties can be used to distinguish one fluorophore from another, and thereby allow for multiplexed analysis. Other desirable properties of the fluorescent label can include cell permeability and low toxicity, for example if labeling of the fusion protein is to be performed in a cell or an organism (e.g., a living animal).

In certain aspects, the conjugated label can comprise an enzyme. Enzymes are desirable labels in some embodiments because amplification of the detectable signal can be obtained and result in increased assay sensitivity. The enzyme itself does not produce a detectable response but functions to break down a substrate when it is contacted by an appropriate substrate such that the converted substrate produces a fluorescent, colorimetric or luminescent signal. Enzymes can amplify the detectable signal because one enzyme on a labeling reagent can result in multiple substrates being converted to a detectable signal. The enzyme substrate is selected to yield the preferred measurable product, e.g. colorimetric, fluorescent or chemiluminescence. Such substrates are extensively used in the art and are well known by one skilled in the art and include for example, oxidoreductases such as horseradish peroxidase and a substrate such as 3,3′-diaminobenzidine (DAB); phosphatase enzymes such as an acid phosphatase, alkaline and a substrate such as 5-bromo-6-chloro-3-indolyl phosphate (BCIP); glycosidases, such as beta-galactosidase, beta-glucuronidase or beta-glucosidase and a substrate such as 5-bromo-4-chloro-3-indolyl beta-D-galactopyranoside (X-gal); additional enzymes include hydrolases such as cholinesterases and peptidases, oxidases such as glucose oxidase and cytochrome oxidases, and reductases for which suitable substrates are known.

Enzymes and their appropriate substrates that produce chemiluminescence are suitable for some assays. These include, but are not limited to, natural and recombinant forms of luciferases and aequorins. Chemiluminescence-producing substrates for phosphatases, glycosidases and oxidases such as those containing stable dioxetanes, luminol, isoluminol and acridinium esters are additionally useful.

In another aspect, haptens, such as biotin, are also utilized as labels. Biotin is useful because it can function in an enzyme system to further amplify the detectable signal, and it can function as a tag to be used in affinity chromatography for isolation purposes. For detection purposes, an enzyme conjugate that has affinity for biotin is used, such as avidin-HRP. Subsequently a peroxidase substrate is added to produce a detectable signal.

Haptens also include hormones, naturally occurring and synthetic drugs, pollutants, allergens, affector molecules, growth factors, chemokines, cytokines, lymphokines, amino acids, peptides, chemical intermediates, nucleotides and the like.

Enzymes and their appropriate substrates that produce chemiluminescence are suitable for some assays. These include, but are not limited to, natural and recombinant forms of luciferases and aequorins. Chemiluminescence-producing substrates for phosphatases, glycosidases and oxidases such as those containing stable dioxetanes, luminol, isoluminol and acridinium esters are additionally useful.

In another aspect, haptens, such as biotin, are also utilized as labels. Biotin is useful because it can function in an enzyme system to further amplify the detectable signal, and it can function as a tag to be used in affinity chromatography for isolation purposes. For detection purposes, an enzyme conjugate that has affinity for biotin is used, such as avidin-HRP. Subsequently a peroxidase substrate is added to produce a detectable signal.

Haptens also include hormones, naturally occurring and synthetic drugs, pollutants, allergens, affector molecules, growth factors, chemokines, cytokines, lymphokines, amino acids, peptides, chemical intermediates, nucleotides and the like.

In certain aspects, fluorescent proteins can be conjugated to the fusion protein as a label. Examples of fluorescent proteins include green fluorescent protein (GFP) and the phycobiliproteins and derivatives thereof. The fluorescent proteins, especially phycobiliprotein, are particularly useful for creating tandem dye labeled labeling reagents. These tandem dyes comprise a fluorescent protein and a fluorophore for the purposes of obtaining a larger stokes shift wherein the emission spectra is farther shifted from the wavelength of the fluorescent protein's absorption spectra.

In certain aspects, the label is a radioactive isotope. Examples of suitable radioactive materials include, but are not limited to, iodine (¹²¹I, ¹²³I, ¹²⁵I, ¹³¹I) carbon (¹⁴C), sulfur (³⁵S), tritium (³H), indium (¹¹¹In, ¹¹²In, ¹¹³mIn, ¹¹⁵mIn), technetium (⁹⁹Tc, ⁹⁹mTc), thallium (²⁰¹Ti), gallium ⁶⁷Ga), palladium (¹⁰³Pd), molybdenum (⁹⁹Mo), xenon (¹³⁵Xe), fluorine (¹⁸F), ¹⁵³Sm, ¹⁷⁷Lu, ¹⁵⁹Gd, ¹⁴⁹Pm, ¹⁴⁰La, ¹⁷⁵Yb, ¹⁶⁶Ho, ⁹⁰Y, ⁴⁷Sc, ¹⁸⁶Re, ¹⁸⁸Re, ¹⁴²Pr, ¹⁰⁵Rh, and ⁹⁷Ru.

In some aspects, drugs (e.g., other active agents) can be conjugated to the fusion protein. For example, a fusion protein can be conjugated to a therapeutic moiety or agent, such as an immunotherapy drug. In some embodiments, the fusion protein can bind to a target cell, and deliver the immunotherapy drug to the cell. Any immunotherapeutic known in the art can be conjugated to the fusion protein. In certain embodiments, drugs and other molecules can be conjugated to a fusion protein via site-specific conjugation. Non-limiting example embodiments of such additional active agents can include antiviral agents, antibiotic agents, antifungal agents, antiparasitic agents, gamma globulin, and the like. Similarly, the fusion protein disclosed herein may be used in combination with one or more other therapeutic interventions that may be used in the treatment of conditions such as lymphopenia and/or immunodeficiency in a subject.

### C. Polynucleotides Encoding Fusion Proteins & Recombinant Cells Producing Fusion Proteins

The disclosure provides methods for producing fusion proteins. In certain aspects, the disclosure provides for recombinant methods of generating a fusion protein. In some embodiments, recombinant nucleic acids encoding for the fusion protein can be operably linked to one or more regulatory nucleotide sequences in an expression construct. In some embodiments, the nucleic acid sequences encoding the signal peptide, first or second polypeptide sequences, or the linker/hinge region can be cloned in the same expression vector in a defined orientation or can be cloned in different vectors. If expression is carried out using one vector, the coding genes can have their own genetic elements (e.g., promoter, RBS, leader, stop, polyA, etc.) or they can be cloned with one single set of genetic elements, but connected with a cistron element. Regulatory nucleotide sequences can be appropriate for a host cell used for expression. Numerous types of appropriate expression vectors and suitable regulatory sequences are known in the art for a variety of host cells. Typically, one or more regulatory nucleotide sequences can include, but are not limited to, promoter sequences, leader or signal sequences, ribosomal binding sites, transcriptional start and termination sequences, translational start and termination sequences, and enhancer or activator sequences. Any known constitutive or inducible promoter are contemplated for use with the aspects and embodiments included in the disclosure. The promoters can be either naturally occurring promoters, or hybrid promoters that combine elements of more than one promoter. An expression construct can be present in a cell on an episome, such as a plasmid, or the expression construct can be inserted in a chromosome.

In certain aspects, an expression vector contains a selectable marker gene to allow the selection of transformed host cells. Selectable marker genes are known and can vary with the host cell used. In certain aspects, the disclosure relates to an expression vector comprising a nucleotide sequence encoding a polypeptide that is operably linked to at least one regulatory sequence. Regulatory sequences are generally known and can be selected to direct expression of the encoded polypeptide. Accordingly, the term regulatory sequence includes promoters, enhancers, and other expression control elements. Exemplary, non-limiting regulatory sequences are described in Goeddel; Gene Expression Technology: Methods in Enzymology, Academic Press, San Diego, CA (1990). It should be understood that the design of the expression vector can depend on such factors as the choice of the host cell to be transformed and/or the type of protein (e.g., fusion protein) to be expressed. Moreover, the vector's copy number, the ability to control that copy number and the expression of any other protein encoded by the vector, such as antibiotic markers, can be considered.

In some embodiments relating to methods of producing a fusion protein, a host cell can be transfected with one or more expression vector(s) encoding a fusion protein, and can be cultured under appropriate conditions to allow expression of the polypeptide(s) to occur. The fusion protein can be secreted and isolated from cells and/or cell culture media containing the fusion protein. Alternatively, the fusion protein can be retained in the cytoplasm or in a membrane fraction and the cells harvested and lysed, and the fusion protein subsequently purified and isolated. A cell culture includes host cells, media and other byproducts. Any suitable media for cell culture can be used in methods of production. Fusion proteins can be isolated from cell culture medium, host cells, or both using common techniques for purifying proteins, including, for example, ion-exchange chromatography, gel filtration chromatography, ultrafiltration, electrophoresis, and immunoaffinity purification. In certain aspects, the fusion protein can be produced with a domain (e.g., a His-tag) that facilitates its purification.

A recombinant nucleic acid can be produced by ligating the cloned gene, or a portion thereof, into a vector suitable for expression in either prokaryotic cells, eukaryotic cells (yeast, avian, insect or mammalian), or both. Expression vehicles for production of a recombinant polypeptide include plasmids and other vectors. For instance, suitable vectors include plasmids of the types: pBR322-derived plasmids, pEMBL-derived plasmids, pEX-derived plasmids, pBTac-derived plasmids and pUC-derived plasmids for expression in prokaryotic cells, such as E. coli. In certain aspects, mammalian expression vectors contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of proteins in eukaryotic cells. The various methods employed in the preparation of the plasmids and transformation of host organisms are well known in the art. For other suitable expression systems for both prokaryotic and eukaryotic cells, as well as general recombinant procedures, see Molecular Cloning A Laboratory Manual, 2nd Ed., ed. by Sambrook, Fritsch and Maniatis (Cold Spring Harbor Laboratory Press, 1989) Chapters 16 and 17. In some instances, it can be desirable to express the recombinant polypeptide(s) by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the ß-gal containing pBlueBac III).

Techniques for making fusion genes are well known. Essentially, the joining of various nucleic acid fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another aspect, the fusion gene can be synthesized by conventional techniques including automated DNA synthesizers. Alternatively, PCR amplification of gene fragments can be carried out using anchor primers which give rise to complementary overhangs between two consecutive nucleic acid fragments which can subsequently be annealed to generate a chimeric gene sequence (see, for example, Current Protocols in Molecular Biology, eds. Ausubel et al., John Wiley & Sons: 1992).

In some aspects, an expression vector expressing any of the nucleic acids described above can be used to express a fusion protein in a host cell. For example, a fusion protein can be expressed in bacterial cells such as E. coli, insect cells (e.g., using a baculovirus expression system), yeast, or mammalian cells. Other suitable host cells are known to those skilled in the art.

Once the expression vector is transferred to a host cell by conventional techniques, the transfected cells are then cultured by conventional techniques to produce a fusion protein. Thus, the disclosure includes host cells containing a polynucleotide encoding a fusion protein, operably linked to a heterologous promoter. In certain aspects, if the fusion protein is encoded from different vectors, the vectors can be co-expressed in the host cell for expression of the entire fusion protein. In certain aspects, the fusion protein is expressed from a single promoter. In certain aspects, the fusion protein is expressed from multiple promoters. In certain aspects, the fusion protein is encoded on a single vector. In certain aspects, the fusion protein is encoded on multiple vectors.

Mammalian cell lines available as hosts for expression of fusion proteins are well known in the art and include many immortalized cell lines available from the American Type Culture Collection (ATCC), including but not limited to Chinese hamster ovary (CHO) cells, HeLa cells, baby hamster kidney (BHK) cells, monkey kidney cells (COS), human hepatocellular carcinoma cells (e.g., Hep G2), human epithelial kidney 293 cells, and a number of other cell lines. Different host cells have characteristic and specific mechanisms for the post-translational processing and modification of proteins and gene products. Appropriate cell lines or host systems can be chosen to ensure the correct modification and processing of the fusion protein or portion thereof expressed. To this end, eukaryotic host cells that possess the cellular machinery for proper processing of the primary transcript, glycosylation, and phosphorylation of the gene product can be used. Such mammalian host cells include but are not limited to CHO, HEK293, VERY, BHK, Hela, COS, MDCK, 293, 3T3, W138, BT483, Hs578T, HTB2, BT2O and T47D, NSO (a murine myeloma cell line that does not endogenously produce any functional immunoglobulin chains), SP20, CRL7O3O and HsS78Bst cells.

In certain aspects, fusion proteins of the disclosure are stably expressed in a cell line. Stable expression can be used for long-term, high-yield production of recombinant proteins. For example, cell lines that stably express the fusion protein can be generated. Host cells can be transformed with an appropriately engineered vector comprising expression control elements (e.g., promoter, enhancer, transcription terminators, polyadenylation sites, etc.), and a selectable marker gene. Following the introduction of the foreign DNA, cells can be allowed to grow for 1-2 days in an enriched media, and then are switched to a selective media. The selectable marker in the recombinant plasmid confers resistance to the selection and allows cells that stably integrated the plasmid into their chromosomes to grow and form foci, which in turn can be cloned and expanded into cell lines. Methods for producing stable cell lines with a high yield are well known in the art and reagents are generally available commercially.

In certain aspects, fusion proteins of the disclosure are transiently expressed in a cell line. Transient transfection is a process in which the nucleic acid introduced into a cell does not integrate into the genome or chromosomal DNA of that cell, but is maintained as an extrachromosomal element, e.g. as an episome, in the cell. Transcription processes of the nucleic acid of the episome are not affected and a protein encoded by the nucleic acid of the episome is produced.

The cell line, either stable or transiently transfected, is maintained in cell culture medium and conditions well known in the art that result in the expression and production of fusion protein. In certain aspects, the mammalian cell culture media is based on commercially available media formulations, including, for example, DMEM or Ham's F12. In other aspects, the cell culture media is modified to support increases in both cell growth and biologic protein expression. As used herein, the terms “cell culture medium,” “culture medium,” and “medium formulation” refer to a nutritive solution for the maintenance, growth, propagation, or expansion of cells in an artificial in vitro environment outside of a multicellular organism or tissue. Cell culture medium can be optimized for a specific cell culture use, including, for example, cell culture growth medium that is formulated to promote cellular growth or cell culture production medium which is formulated to promote recombinant protein production. The terms “nutrient”, “ingredient”, and “component” are used interchangeably herein to refer to the constituents that make up a cell culture medium.

Once a fusion protein has been produced, it can be purified by any method known in the art for purification of a protein complex, for example, by chromatography (e.g., ion exchange, affinity, and size column chromatography), centrifugation, differential solubility, or by any other standard technique for the purification of proteins.

When using recombinant techniques, the fusion protein can be produced intracellularly, in the periplasmic space, or directly secreted into the medium. If the protein is produced intracellularly, as a first step, the particulate debris, either host cells or lysed fragments, is removed, for example, by centrifugation or ultrafiltration. Where the protein is secreted into the medium, supernatants from such expression systems are generally first concentrated using a commercially available protein concentration filter, for example, an Amicon or Millipore Pellicon ultrafiltration unit. A protease inhibitor such as PMSF can be included in any of the foregoing steps to inhibit proteolysis and antibiotics can be included to prevent the growth of adventitious contaminants.

The composition prepared from the cells can be purified using, for example, hydroxylapatite chromatography, hydrophobic interaction chromatography, ion exchange chromatography, gel electrophoresis, dialysis, and/or affinity chromatography either alone or in combination with other purification steps. The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Other techniques for protein purification such as fractionation on an ion-exchange column, ethanol precipitation, Reverse Phase HPLC, chromatography on silica, chromatography on heparin, SEPHAROSE chromatography on an anion or cation exchange resin (such as a polyaspartic acid column), chromatofocusing, SDS-PAGE, and ammonium sulfate precipitation are also available.

Regardless of how a fusion protein is purified, to confirm functional binding activity, binding assays can be performed (before and/or after purification). For example, ELISA assays, including dual ELISA assays can be used. In some aspects, a first binding target is coated on a well, and binding to this target immobilizes the fusion protein. A tagged second binding target is added to the well, and detected. Only fusion proteins that are both immobilized via binding to the first binding target and bound to the second binding target will be detected.

In some aspects, the disclosure provides for recombinant cell lines expressing the fusion protein that can be deposited and maintained with an international depository institution that is authorized under the provisions of the Budapest Treaty (i.e., an International Depositary Authority, IDA).

### D. Pharmaceutical Formulations

In certain aspects, the disclosure provides pharmaceutical compositions. Such pharmaceutical compositions can also be compositions comprising a fusion protein as disclosed herein and a pharmaceutically acceptable excipient. In certain aspects, the pharmaceutical compositions of the disclosure are used as a medicament (i.e., in methods of treating or preventing a disease or condition (e.g., lymphopenia), in a subject in need of treatment or preventative treatment). In some embodiments, pharmaceutical compositions can be compositions comprising a nucleic acid molecule that encodes a fusion protein as disclosed herein.

In certain aspects, a fusion protein (or a polynucleotide encoding the fusion protein) can be formulated with a pharmaceutically acceptable carrier, excipient or stabilizer, as pharmaceutical compositions. In certain aspects, such pharmaceutical compositions are suitable for administration to a human, or a non-human mammal or animal, via any one or more route of administration using methods known in the art. The route and/or mode of administration will vary depending upon the desired results. The term “pharmaceutically acceptable carrier” means one or more non-toxic materials that do not interfere with the effectiveness of the biological activity of the active ingredients. Such preparations can routinely contain salts, buffering agents, preservatives, compatible carriers, and optionally other therapeutic agents. Such pharmaceutically acceptable preparations can also contain compatible solid or liquid fillers, diluents or encapsulating substances, which are suitable for administration into a human. Other contemplated carriers, excipients, and/or additives, which can be utilized in the formulations described herein include, for example, flavoring agents, antimicrobial agents, sweeteners, antioxidants, antistatic agents, lipids, protein excipients such as serum albumin, gelatin, casein, salt-forming counterions such as sodium and the like. These and additional known pharmaceutical carriers, excipients and/or additives suitable for use in the formulations described herein are known in the art, e.g., as listed in “Remington: The Science & Practice of Pharmacy”, 21^{st} ed., Lippincott Williams & Wilkins, (2005), and in the “Physician's Desk Reference”, 60^{th} ed., Medical Economics, Montvale, N.J. (2005). Pharmaceutically acceptable carriers can be selected that are suitable for the mode of administration, solubility and/or stability desired or required.

The formulations described herein comprise active agents (i.e., one or more fusion proteins as disclosed herein) in a concentration resulting in a w/v appropriate for a desired dose. In certain aspects, the active agent is present in a formulation at a concentration of about 1 mg/ml to about 200 mg/ml, about 1 mg/ml to about 100 mg/ml, about 1 mg/ml to about 50 mg/ml, or about 1 mg/ml to about 25 mg/ml. In certain aspects, the concentration of the active agent in a formulation can vary from about 0.1% to about 75% by total weight. In certain aspects, the concentration of the active agent is in the range of 0.003 to 1.0 molar.

When used for in vivo administration, the formulations should be sterile. Formulations can be sterilized by various sterilization methods, including sterile filtration, radiation, etc. In one aspect, the formulation is filter-sterilized with a presterilized 0.22-micron filter. Sterile compositions for injection can be formulated according to conventional pharmaceutical practice as described in “Remington: The Science & Practice of Pharmacy”, 21^{st} ed., Lippincott Williams & Wilkins, (2005).

Therapeutic compositions are encompassed by the pharmaceutical formulations described herein, and can formulated for particular routes of administration, such as oral, nasal, pulmonary, topical (including buccal and sublingual), rectal, vaginal and/or parenteral administration. The phrases “parenteral administration” and “administered parenterally” as used herein refer to modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

The formulations can be in unit dosage form and prepared by any known method. Actual dosage levels of the active ingredients in the pharmaceutical compositions can be varied to obtain an amount of the active ingredient that is effective to achieve the desired therapeutic response for a particular patient, composition, and mode of administration, without being toxic to the patient (e.g., “a therapeutically effective amount”). The selected dosage level will depend upon a variety of pharmacokinetic factors including the activity of the particular compositions employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts. Suitable dosages can range from about 0.0001 to about 100 mg/kg of body weight or greater, for example about 0.1, 1, 10, or 50 mg/kg of body weight, with about 1 to about 10 mg/kg of body weight being suitable.

In some embodiments of the disclosure, the formulations can be suitable for research use. The concentration of active agent in such formulations, as well as the presence or absence of excipients and/or pyrogens, can be modified or selected based on the particular application and intended use.

The fusion proteins described herein can be used in methods that prevent, treat, or mitigate lymphopenia or immunodeficiency in a subject. As used herein, “lymphopenia”, “lymphocytic leukopenia”, or “lymphocytopenia” refer to a condition characterized by a reduced number of lymphocytes (i.e. T lymphocytes (aka T cells), B lymphocytes (aka B cells), and/or natural killer cells (aka NK cells)) in a subject's blood. As used herein, “immunodeficiency” and “immunodeficient” refer to a physiological state in which a subject's immune system is compromised in such a way that prevents an adequate immune response to fight infection and disease. In some aspects, immunodeficiency increases the subject's susceptibility to microbial infection (e.g. bacterial, viral, fungal, or protozoan infection).

In some aspects, lymphopenia in an adult subject is characterized by a lymphocyte count of less than 1,000-1,500 lymphocytes/uL blood. In some aspects, lymphopenia in a pediatric or adolescent subject is characterized by a lymphocyte count of less than 3,000 lymphocytes/uL blood. Assays to measure lymphocyte counts are known in the art and include complete blood count (CBC), flow cytometry, mitogen- and antigen-induced lymphocyte proliferation assays and ELISA. In some aspects, the fusion proteins of the disclosure can be used to increase white blood counts, increase lymphocyte counts, and/or increase T cell subpopulations in a subject in need thereof. In other aspects, the fusion proteins can be used to protect an immunocompromised subject or a subject at risk of becoming immunocompromised from developing lymphopenia or immunodeficiency. As used herein, “immunocompromised” refers to a subject having a weakened immune system or a reduced ability to fight infections or other diseases, due to a genetic disorder or disease, an infection, an environmental disorder or disease, or other environmental factors. Such methods can be carried out by administering to the patient a therapeutically effective dose of the fusion protein of the disclosure, or pharmaceutical compositions comprising such fusion proteins.

In some aspects, the disclosure relates to methods of treating or preventing lymphopenia or immunodeficiency in a subject having a disease or condition including, without limitation, cancer, human immunodeficiency virus (HIV), hepatitis B, hepatitis C, sepsis, aplastic anemia, lymphoma, an inherited immune disorder, tuberculosis, renal failure, autoimmune disorders such as lupus, rheumatoid arthritis, and myasthenia gravis, and heart failure. Methods of treatment and/or prevention of lymphopenia or immunodeficiency also include subjects receiving chemotherapy, radiation treatment, steroids, aderenocorticotropic hormone, and/or immunosuppressive medications as well as those having undergone stem cell, tissue, and/or organ transplantation.

As used herein, the term “disease” refers to any pathological state resulting in lymphopenia or immunodeficiency or a pathological state that may increase a subject's susceptibility to developing lymphopenia or immunodeficiency.

As used herein, lymphopenia or immunodeficiency can be determined or identified based on any available and/or accepted test to diagnose such conditions, including clinical presentation (i.e. analysis of physical signs or symptoms), blood count, and/or measurement of lymphocyte subpopulations and immunoglobulin levels. In some aspects, lymphopenia in a subject can be asymptomatic. In other aspects, symptoms of lymphopenia can include absent or diminished tonsils or lymph nodes, skin abnormalities (e.g. alopecia, eczema, pyoderma, and telangiectasia), evidence of hematologic disorder (e.g. pallor, petechiae, jaundice, and mouth ulcers), lymphadenopathy, splenomegaly, and recurring infection.

As used herein, “treat”, “treating”, or “treatment” refer to administering a fusion protein or composition as described herein to a subject in order to eliminate, reduce, or attenuate the clinical symptoms of lymphopenia or immunodeficiency; arrest, inhibit, reverse, or slow the progression of lymphopenia or immunodeficiency in a subject; prevent, reverse, or slow the loss of lymphocyte counts in the blood of a subject; stimulate the recovery of lymphocyte counts in the blood of a subject; stimulate the proliferation and/or differentiation of lymphocytes in a subject; and/or stimulate the proliferation and/or differentiation of white blood cells in a subject.

In some aspects, the disclosure provides methods for preventing lymphopenia or immunodeficiency in a subject. In some further aspects of the methods relating to preventing disease, the subject can be at risk for developing lymphopenia or immunodeficiency. By “being at risk” or having an “increased risk”, a subject is identified as having a higher than normal chance of developing lymphopenia or immunodeficiency as compared to the general population. In some embodiments, a subject who has had, or who currently has cancer, HIV, hepatitis B, hepatitis C, sepsis, aplastic anemia, lymphoma, an inherited immune disorder, tuberculosis, renal failure, autoimmune disorders such as lupus, rheumatoid arthritis, and myasthenia gravis, heart failure, or is otherwise considered susceptible to being or becoming immunocompromised has an increased risk of developing lymphopenia or immunodeficiency. At risk subjects can also include subjects receiving chemotherapy, radiation treatment, steroids, aderenocorticotropic hormone, and/or immunosuppressive medications as well as those having undergone stem cell, tissue, and/or organ transplantation.

The term “immunotherapy” relates to a treatment involving a specific immune response, specifically, stimulation or recovery of immune cell populations. In the context of the present disclosure, terms such as “protect”, “prevent”, “prophylactic”, “preventative”, or “protective” relate to the prevention of the occurrence of or the exacerbation of lymphopenia or immunodeficiency in a subject. Persons having lymphopenia or immunodeficiency, immunocompromised persons, or persons at risk of developing lymphopenia, immunodeficiency, or becoming immunocompromised, as described above, would be considered candidates for immunotherapy.

A prophylactic administration of an immunotherapy, for example, a prophylactic administration of a fusion protein or a composition comprising a fusion protein as disclosed herein, can in certain embodiments protect the recipient from developing lymphopenia or immunodeficiency. In some embodiments, prophylactic administration can reduce the severity of lymphopenia or immunodeficiency.

A therapeutic administration of an immunotherapy, for example, a therapeutic administration of a fusion protein or a composition comprising a fusion protein as disclosed herein, can inhibit or attenuate the progression of lymphopenia, arrest or recover loss of white blood cell and/or lymphocyte counts, and/or inhibit, reduce, arrest, or alleviate clinical symptoms of lymphopenia.

The terms “subject”, “individual”, or “patient” are interchangeable, and relate to vertebrates, preferably mammals. For example, mammals in the context of the disclosure are humans, non-human primates, domesticated animals such as dogs, cats, sheep, cattle, goats, pigs, horses etc., laboratory animals such as mice, rats, rabbits, guinea pigs, etc., as well as animals in captivity such as animals in zoos. The term “animal” as used herein also includes humans. The term “subject” can also include a patient, i.e., an animal, an in particular embodiments, a human having lymphopenia, immunodeficiency, or an immunocompromised person.

The fusion proteins and compositions described herein can be administered via any conventional route, including by injection or infusion. In some embodiments, the administration can be carried out, for example, orally, intravenously, intraperitonealy, intramuscularly, subcutaneously, or transdermally.

The fusion proteins and the compositions comprising them are administered in effective amounts. An “effective amount” includes an amount that achieves a desired reaction or a desired effect and can be in the form of a single dose or as multiple doses. In the case of treatment of lymphopenia or immunodeficiency, the desired reaction relates to inhibition or attenuation of lymphopenia; recovery, interruption, or prevention of further loss of immune cell populations (e.g. white blood cells and lymphocytes); and/or alleviation or attenuation of clinical symptoms. A desired reaction can also be delay of onset or prevention of onset of lymphopenia or immunodeficiency.

An effective amount of a composition of the disclosure will depend on the severity of lymphopenia or immunodeficiency, underlying cause (i.e. disease or condition) of lymphopenia or immunodeficiency, the individual parameters of the patient, including age, physiological condition, size and weight, the duration of treatment, the type of an accompanying therapy (if present), the specific route of administration and similar factors. Accordingly, the doses of the compositions of the disclosure administered can depend on various combinations of such parameters. In embodiments in which an initial amount administered to a patient is insufficient, further administration with higher amounts, more frequent doses, or a different/more localized route of administration can be used.

In the methods and compositions disclosed herein, an IL-7 fusion protein can be administered to a subject to extend the serum half-life of the IL-7 protein relative to recombinant or exogenous IL-7. In certain embodiments, the half-life of the IL-7 protein is extended by at least 2 hours, more preferably by at least 4 hours when administered to a subject.

In some embodiments, when a subject is administered a fusion protein in a range of 50-150 μg/kg, the serum half-life of the human IL-7 is about 190-230 minutes, more preferably 190-330 minutes, relative to recombinant or exogenous IL-7.

In some embodiments, the IL-7 half-life extension of fusion proteins as described herein is sufficient to stimulate white blood cell proliferation, lymphocyte proliferation, and/or lymphocyte differentiation in a subject.

In certain embodiments, the IL-7 half-life extension of fusion proteins as described herein is sufficient to stimulate an increase in lymphocyte counts, specifically T cell counts, and more specifically T cell subpopulations including CD3+CD4⁺ T cells, CD3+CD4⁺ T cells, and CD3⁺ T cells.

Recovery or stimulation of lymphocyte populations can be facilitated by binding of the fusion protein with the IL-7 receptor of target immune cells.

### F. Kits

Another aspect of the present disclosure is a kit. In one aspect, a kit comprises any of the compositions or pharmaceutical compositions of a nucleic acid, polypeptide, expression vector, or host cell described above, and instructions or a label directing appropriate use or administration. Optionally, a kit can also include one or more containers and/or a syringe or other device to facilitate delivery or use. The disclosure contemplates that all or any subset of the components for conducting research assays and/or for administering therapeutically effective amounts of a fusion protein can be enclosed in the kit. Similarly, the kit can include instructions for making a polypeptide by, for example culturing a host cell that expresses a nucleic acid that encodes a fusion protein of the disclosure under suitable conditions. By way of additional example, a kit for therapeutic administration of a fusion protein of the disclosure can comprise a solution containing a pharmaceutical formulation of the fusion protein, or a lyophilized preparation of the fusion protein, and instructions for administering the composition to a patient in need thereof and/or for reconstituting the lyophilized product.

The present disclosure also encompasses a finished packaged and labeled pharmaceutical product. This article of manufacture includes the appropriate unit dosage form in an appropriate vessel or container such as a glass vial or other container that is hermetically sealed. In the case of dosage forms suitable for parenteral administration the active ingredient, e.g., an above-described fusion protein, is sterile and suitable for administration as a particulate free solution. In certain aspects, the formulation is suitable for intravenous administration, such as for intravenous infusion to a human or animal.

In a specific aspect, the formulations of the disclosure are formulated in single dose vials as a sterile liquid. Exemplary containers include, but are not limited to, vials, bottles, pre-filled syringes, IV bags, and blister packs (comprising one or more pills). Optionally associated with such container(s) can be a notice in the form prescribed by a governmental agency regulating the manufacture, use or sale of pharmaceuticals or biological products, which notice reflects approval by the agency of manufacture, use or sale for human diagnosis and/or administration.

As with any pharmaceutical product, the packaging material and container are designed to protect the stability of the product during storage and shipment. Further, the products of the disclosure include instructions for use or other informational material that advise the physician, technician, or patient on how to appropriately prevent or treat the disease or disorder in question. In other words, the article of manufacture includes instruction means indicating or suggesting a dosing regimen including, but not limited to, actual doses, monitoring procedures, etc., and other monitoring information.

A kit for research purposes can comprise a solution containing a fusion protein or a lyophilized preparation of a fusion protein of the disclosure, wherein the fusion protein binds specifically to one or more targets. The fusion protein can be labeled according to methods known in the art and described herein, including but not limited to labels such as small molecule fluorescent tags, proteins such as biotin, GFP or other fluorescent proteins, or epitope sequences such as his or myc. Similarly, primary antibodies used for detecting the fusion protein can be included in the kit. Primary antibodies can be directed to sequences on the fusion protein or to labels, tags, or epitopes with which the fusion protein is labeled. Primary antibodies can, in turn, be labeled for detection, or, if further amplification of the signal is desired, the primary antibodies can be detected by secondary antibodies, which can also be included in the kit. In some aspects, kits for research use can resemble kits intended therapeutic uses but further include a label specifying that the kit and its use is restricted to research purposes only.

G. Modification and/or Engineering

The disclosed fusion proteins can also be used to produce therapeutic immunoconjugates, wherein the fusion protein is conjugated with one or more therapeutic agents. For example, fusion proteins as described herein can be used in the production of protein-drug conjugates. Drugs that can be used include, without limitation, large and small molecule active agents such as, for example, immune system modulators, growth factors, (e.g., colony-stimulating factors (CSFs)), antiviral agents, antibiotic agents, antifungal agents, antiparasitic agents, and gamma globulin, and the like.

### EXAMPLES

### Example 1: Amplification of IL-7 Fusion Proteins

Tables 1 and 2 detail source DNA and primers for Polymerase Chain Reaction (PCR) amplification of exemplary human IL-7 (hIL-7), HSA, signal peptide, and hinge peptide in accordance with example embodiments as disclosed herein.

TABLE 1
Source DNA for Representative hIL-7, HSA,
Signal Peptide, and Hinge Peptide

| | Source |
|---|---|
| DNA | (UniProt Knowledgebase ID) |
| Human IL-7 | P13232 |
| HSA | P02768 |
| Mouse IgG kappa light chain | GenBank: AAH80787.1 |
| (signal peptide) | |
| Human IgD (hinge peptide) | P0DOX3 |

TABLE 2
PCR Primers for Amplification of Representative hIL-7, HSA,
Signal Peptide, and Hinge Peptide

| Target | Primers |
|---|---|
| Human IL-7 | 5′ GTGCCTGGCAGCACCGGAGATTGTGATATTGAGGGC 3′ |
| (JL18008-1) | (SEQ ID NO: 1) |
| | 5′ GCCTGGGCCTTTGGGGACTCGTGTTCCTTTGTGCCCATC 3′ |
| | (SEQ ID NO: 2) |
| Human IL-7 | 5′ AGAGAGACCAAGACACCAGATTGTGATATTGAGGGCA 3′ |
| (JL18008-2) | (SEQ ID NO: 3) |
| | 5′ ACGGGCCCTCTAGACTCGAGTCATCAGTGTTCCTTTGTGCCCATCAG 3′ |
| | (SEQ ID NO: 4) |
| HSA (JL18008-1) | 5′ AGAGAGACCAAGACACCAGATGCTCACAAGTCTGAGG 3′ |
| | (SEQ ID NO: 5) |
| | 5′ ACGGGCCCTCTAGACTCGAGTCATCACAGGCCCAGGGCAGCCTG 3′ |
| | (SEQ ID NO: 6) |
| HSA (JL18008-2) | 5′ GTGCCTGGCAGCACCGGAGATGCTCACAAGTCTGAG 3′ |
| | (SEQ ID NO: 7) |
| | 5′ GCCTGGGCCTTTGGGGACTCCAGGCCCAGGGCAGCCTG 3′ |
| | (SEQ ID NO: 8) |
| Mouse IgG kappa | 5′ CGTTTAAACTTAAGCTTGGTACCGAGC 3′ |
| light chain (signal | (SEQ ID NO: 9) |
| peptide) | 5′ TCCGGTGCTGCCAGGCACCC 3′ |
| | (SEQ ID NO: 10) |
| Human IgD | 5′ GAGTCCCCAAAGGCCCAGGC 3′ |
| (hinge peptide) | (SEQ ID NO: 11) |
| | 5′ TGGTGTCTTGGTCTCTCTCTC 3′ |
| | (SEQ ID NO: 12) |

PCR may be performed according to typical protocols, and may include or be adapted from the following strategy. HSA-IL7 is assembled by overlapping PCR techniques from four DNA fragments that encode for mouse IgG kappa light chain signal peptide, human HSA, the IgD hinge, and human interleukin-7. The individual DNA fragments corresponding to each of the above-mentioned genes are amplified by PCR in a 50 ul reaction prepared by mixing 2.5 ul of 10 uM forward primer, 2.5 ul 10 uM reverse primer, 1 ul diluted DNA template, 19 ul deionized water, 25 ul Q5 High Fidelity 2× Master Mix (M0492, NEB). The reaction is performed by preheating at 98° C. for 30 seconds, then 25 cycles of denaturation at 98° C. for 10 seconds, annealing at 65° C. for 30 seconds, and extension at 72° C. for 60 seconds. The PCR products are separated on a 1.2% agarose gel and are purified using gel DNA extraction kit (D4002, Zymo Research). A 2-step assembly protocol is used by assembling the signal peptide with HSA and the hinge peptide with IL-7, respectively, in a first step. The resulting PCR products are further assembled to give rise to the HSA-IL7 fusion gene. The assembly reaction comprises the pre-assembly reaction and the amplification reaction. The pre-assembly reaction is set up in a 30 ul reaction by mixing the two purified PCR products with 15 ul Q5 High Fidelity 2× Master Mix, and by running between 98° C. (10 sec) and 68° C. (80 sec) for 7 cycles. The amplification reaction is set up by adding 3 ul 10 uM forward primer, 3 ul 10 uM reverse primer, 9 ul dH₂O, and 15 ul Q5 High Fidelity 2× Master Mix to the 30 ul pre-assembly reaction. The reaction utilizes a regular PCR protocol with preheating at 98° C. for 30 seconds and with 30 cycles of denaturation at 98° C. for 10 seconds, annealing at 55° C. for 30 seconds, and extension at 72° C. for 90 seconds. The final PCR product is digested by HindIII and XbaI, and is subcloned into the mammalian expression vectors pJL115 under a human pEF1 promoter. The coding region is confirmed by DNA sequencing (Genewiz). The human EF1 promoter from pEFlalpha-IRES containing a single point mutation (T578C) is amplified using the following primers: pCDNA3.1 hEF1a F1 5′ CGCGCGACGCGTGGCTCCGGTGCCCGTCA 3′ (SEQ ID NO: 13) pCDNA3.1 hEF1a R1 5′ CTACTAGCTAGCTCACGACACCTGAAATGGAAGAAA 3′ (SEQ ID NO: 14). Amplification reactions are performed with NEB Q5 2× Master Mix. The purified PCR products and pCDNA3.1(+) vector are digested with MluI at 37° C. for 1.5 hours. The PCR product(s) and vector are further digested with NheI at 37° C. for 1.5 hours. Ligation of the digested products is performed using T4 DNA ligase with an insert:vector molar ratio of 3:1. Ligation is carried out at 25° C. for 1 hour.

### Example 2: Transfection and Cell Line Generation

Cell lines are generated via transfection of CHO K1 cells (ATCC) with a plasmid carrying a JL18008-1 or JL18008-2 expression cassette. Plasmid DNA is linearized by ScaI (NEB, Cat #R3122S), purified by Phase Lock gel (5Prime, Cat #2302830), and transfected using Freestyle Max (Gibco, Cat #94764) according to manufacturer's instruction. After transfection, the bulk pools and mini pools are generated from 0.5×10⁶ cells and 5000 cells, respectively, in the presence of 12.5 μM MSX. The recovered pools are assessed using IL-7 ELISA kit (PEPROTECH Human IL-7 Standard ABTS ELISA Development Kit, Cat #900-K17) and top mini pools are subjected to two rounds of FACS cloning. FACS sortings are performed by staining cells with FITC-conjugated anti-HSA antibody made in house using a labeling kit (Sigma, Cat #FITC-1KT). 960 clones are deposited into 96-well plates and grown in CD CHO medium containing 1×HT supplement (Gibco, Cat #11067-030) and ClonaCell-CHO ACF Supplement (Stemcell, Cat #03820). After recovery from selection, top clones are gradually scaled up to shake flask. The titer is determined by Octet measurement using SAX sensors coated with CaptureSelect Biotin Anti-HSA Conjugate (Thermo Fisher, Cat #7102972100). Single-cell clones are generated by limiting-dilution cloning.

Transfected cells are cultured for 7-14 days to produce conditioned media. Conditioned media is collected and treated by centrifugation and/or filtration to remove cellular debris. JL18008-1 and JL18008-2 fusion proteins are isolated and purified from the conditioned media by a series of column chromatography, ultra filtration, and diafiltration. Column chromatography is performed using Capto Blue (GE Healthcare), Toyopearl Phenyl-650M (Tosoh Bioscience), Capto G HP (GE Healthcare), Capto SP ImpRes (GE Healthcare), and/or CaptureSelect (Thermo Fisher).

Tables 3 and 4 detail sequence information for exemplary IL-7 fusion proteins in accordance with example embodiments.

TABLE 3
Amino Acid Sequences for Representative Protein and Peptide Sequences

| Protein/Peptide | Sequence |
|---|---|
| human IL-7 | DCDIEGKDGKQYESVLMVSIDQLLDSMKEIGSNCLNN |
| | EFNFFKRHICDANKEGMFLFRAARKLRQFLKMNSTG |
| | DFDLHLLKVSEGTTILLNCTGQVKGRKPAALGEAQPT |
| | KSLEENKSLKEQKKLNDLCFLKRLLQEIKTCWNKILM |
| | GTKEH (SEQ ID NO: 15) |
| Human Serum Albumin | DAHKSEVAHRFKDLGEENFKALVLIAFAQYLQQCPFE |
| (HSA) | DHVKLVNEVTEFAKTCVADESAENCDKSLHTLFGDK |
| | LCTVATLRETYGEMADCCAKQEPERNECFLQHKDDN |
| | PNLPRLVRPEVDVMCTAFHDNEETFLKKYLYEIARRH |
| | PYFYAPELLFFAKRYKAAFTECCQAADKAACLLPKLD |
| | ELRDEGKASSAKQRLKCASLQKFGERAFKAWAVARL |
| | SQRFPKAEFAEVSKLVTDLTKVHTECCHGDLLECADD |
| | RADLAKYICENQDSISSKLKECCEKPLLEKSHCIAEVE |
| | NDEMPADLPSLAADFVESKDVCKNYAEAKDVFLGMF |
| | LYEYARRHPDYSVVLLLRLAKTYETTLEKCCAAADPH |
| | ECYAKVFDEFKPLVEEPQNLIKQNCELFEQLGEYKFQN |
| | ALLVRYTKKVPQVSTPTLVEVSRNLGKVGSKCCKHPE |
| | AKRMPCAEDYLSVVLNQLCVLHEKTPVSDRVTKCCT |
| | ESLVNRRPCFSALEVDETYVPKEFNAETFTFHADICTL |
| | SEKERQIKKQTALVELVKHKPKATKEQLKAVMDDFA |
| | AFVEKCCKADDKETCFAEEGKKLVAASQAALGL |
| | (SEQ ID NO: 16) |
| Mouse IgG kappa light chain | METDTLLLWVLLLWVPGSTG (SEQ ID NO: 17) |
| (signal peptide) | |
| Human IgD (hinge peptide) | ESPKAQASSVPTAQPQAEGSLAKATTAPATTRNTGRG |
| | GEEKKKEKEKEEQEERETKTP (SEQ ID NO: 18) |

TABLE 4
Amino Acid Sequences for Representative IL-7 Fusion Proteins

| IL-7 Fusion Protein | |
|---|---|
| (Internal Reference) | Sequence |
| JL18008-1 (IL7-HSA) | METDTLLLWVLLLWVPGSTGDCDIEGKDGKQYESVL |
| | MVSIDQLLDSMKEIGSNCLNNEFNFFKRHICDANKE |
| | GMFLFRAARKLRQFLKMNSTGDFDLHLLKVSEGTTIL |
| | LNCTGQVKGRKPAALGEAQPTKSLEENKSLKEQKKL |
| | NDLCFLKRLLQEIKTCWNKILMGTKEHESPKAQASS |
| | VPTAQPQAEGSLAKATTAPATTRNTGRGGEEKKKE |
| | KEKEEQEERETKTPDAHKSEVAHRFKDLGEENFKAL |
| | VLIAFAQYLQQCPFEDHVKLVNEVTEFAKTCVADES |
| | AENCDKSLHTLFGDKLCTVATLRETYGEMADCCAKQ |
| | EPERNECFLQHKDDNPNLPRLVRPEVDVMCTAFHD |
| | NEETFLKKYLYEIARRHPYFYAPELLFFAKRYKAAFTE |
| | CCQAADKAACLLPKLDELRDEGKASSAKQRLKCASL |
| | QKFGERAFKAWAVARLSQRFPKAEFAEVSKLVTDL |
| | TKVHTECCHGDLLECADDRADLAKYICENQDSISSK |
| | LKECCEKPLLEKSHCIAEVENDEMPADLPSLAADFV |
| | ESKDVCKNYAEAKDVFLGMFLYEYARRHPDYSVVL |
| | LLRLAKTYETTLEKCCAAADPHECYAKVFDEFKPLVE |
| | NLIKQNCELFEQLGEYKFQNALLVRYTKKVPQVSTP |
| | TLVEVSRNLGKVGSKCCKHPEAKRMPCAEDYLSVV |
| | LNQLCVLHEKTPVSDRVTKCCTESLVNRRPCFSALE |
| | VDETYVPKEFNAETFTFHADICTLSEKERQIKKQTA |
| | LVELVKHKPKATKEQLKAVMDDFAAFVEKCCKADD |
| | KETCFAEEGKKLVAASQAALGL (SEQ ID NO: 19) |
| JL18008-2 (HSA-IL7) | METDTLLLWVLLLWVPGSTGDAHKSEVAHRFKDLGEEN |
| | FKALVLIAFAQYLQQCPFEDHVKLVNEVTEFAKTCVADES |
| | AENCDKSLHTLFGDKLCTVATLRETYGEMADCCAKQEPE |
| | RNECFLQHKDDNPNLPRLVRPEVDVMCTAFHDNEETFLK |
| | KYLYEIARRHPYFYAPELLFFAKRYKAAFTECCQAADKAAC |
| | LLPKLDELRDEGKASSAKQRLKCASLQKFGERAFKAWAVA |
| | RLSQRFPKAEFAEVSKLVTDLTKVHTECCHGDLLECADDR |
| | ADLAKYICENQDSISSKLKECCEKPLLEKSHCIAEVENDEMP |
| | ADLPSLAADFVESKDVCKNYAEAKDVFLGMFLYEYARRHP |
| | DYSVVLLLRLAKTYETTLEKCCAAADPHECYAKVFDEFKPL |
| | VEEPQNLIKQNCELFEQLGEYKFQNALLVRYTKKVPQVST |
| | PTLVEVSRNLGKVGSKCCKHPEAKRMPCAEDYLSVVLNQ |
| | LCVLHEKTPVSDRVTKCCTESLVNRRPCFSALEVDETYVPK |
| | EFNAETFTFHADICTLSEKERQIKKQTALVELVKHKPKATK |
| | EQLKAVMDDFAAFVEKCCKADDKETCFAEEGKKLVAAS |
| | QAALGLESPKAQASSVPTAQPQAEGSLAKATTAPATTRN |
| | TGRGGEEKKKEKEKEEQEERETKTPDCDIEGKDGKQYESV |
| | LMVSIDQLLDSMKEIGSNCLNNEFNFFKRHICDANKEGM |
| | FLFRAARKLRQFLKMNSTGDFDLHLLKVSEGTTILLNCTG |
| | QVKGRKPAALGEAQPTKSLEENKSLKEQKKLNDLCFLKRL |
| | LQEIKTCWNKILMGTKEH (SEQ ID NO: 20) |

### Example 3: Pharmacokinetic Profiles of IL-7 Fusion Proteins

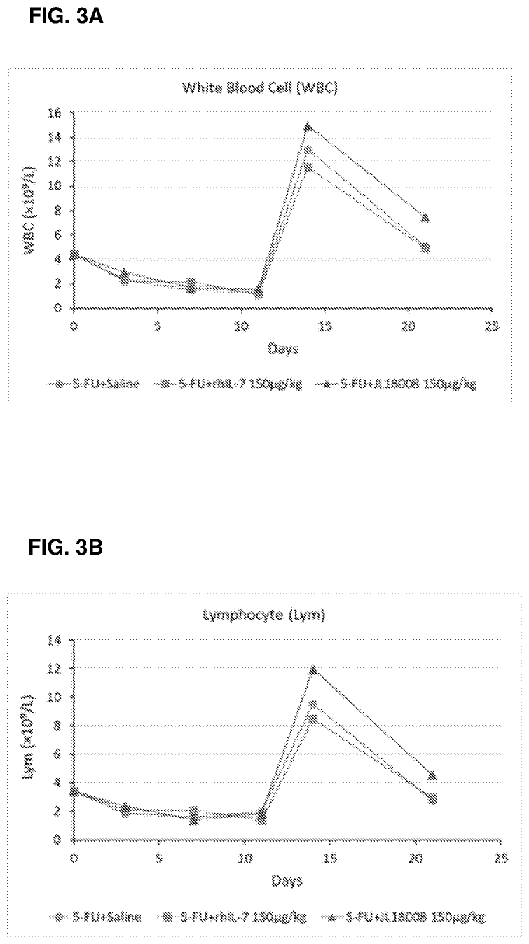

The pharmacokinetic (PK) profiles of JL18008-1 (IL-7-HSA) and JL18008-2 (HSA-IL-7) fusion proteins are examined. JL18008-1 and JL18008-2 are diluted in saline and administered intravenously to C57BL6/N mice at 50 mg/kg or 150 mg/kg. Recombinant human IL-7 (rhIL-7; Peprotech) is administered as a control at 150 mg/kg. Blood samples are obtained by retro-orbital bleeding at 5 min, 30 min, 1 hr, 2 hr, 4 hr, 8 hr, 24 hr, 72 hr, 96 hr, 120 hr, and 192 hr post-injection. The blood samples are centrifuged (5000 rpm) at 4° C. for 15 min to obtain serum. The concentration of human IL-7 (hIL-7) in JL18008-1, JL18008-2, and rhIL-7 serum samples are analyzed by Human IL-7 Quantikine HS ELISA Kit (R&D Systems, Cat #HS750), and PK values are calculated with PKSolver software. PK values are summarized in Table 5 and illustrated in FIG. 2.

TABLE 5
PK Profile of JL18008-1 and JL18008-2

| | Dose | Cₘₐₓ | Tₘₐₓ | T_{1/2} | AUC₀₋ₜ |
|---|---|---|---|---|---|
| Sample | (μg/kg) | (ng/mL) | (min) | (min) | (ng/ml*min) |
| JL18008-1 | 150 | 285.54 | 5 | 323.64 | 64790.32 |
| (IL-7-HSA) | 50 | 81.46 | 5 | 199.40 | 14758.73 |
| JL18008-2 | 150 | 203.69 | 5 | 220.55 | 52785.14 |
| (HSA-IL-7) | 50 | 134.04 | 5 | 195.83 | 16233.22 |
| rhIL-7 (PeproTech) | 150 | 76.33 | 5 | 68.56 | 4108.28 |

At 150 mg/kg, rhIL-7 exhibits a half-life of 68.56 min (˜1.1 hour). At 50 mg/kg and 150 mg/kg, JL18008-1 exhibits a half-life of 323.54 min (5.4 hr) and 199.40 min (3.3 hr), respectively. At 50 mg/kg and 150 mg/kg, JL18008-2 exhibits a half-life of 220.55 min (3.7 hr) and 195.83 min (3.3 hr), respectively. These data demonstrate the IL-7 fusion proteins extend the half-life of hIL-7 as compared to control rhIL-7.

### Example 4: Effect of JL18008-2 on Lymphopenic Mice

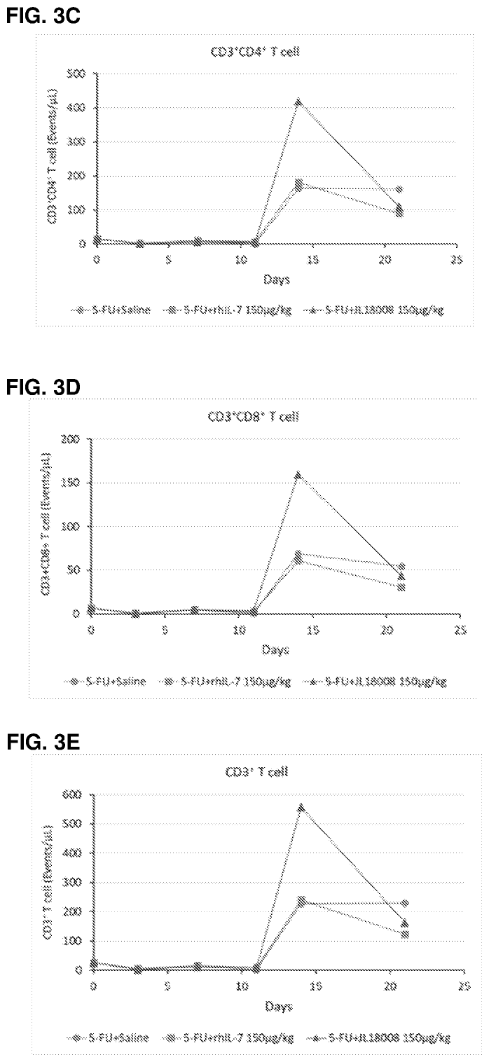

The efficacy of JL18008-2 (HSA-IL-7) is examined in vivo using a murine model of lymphopenia. Balb/C mice are injected intraperitoneally with 200 mg/kg of the chemotherapy drug 5-fluorouracil (5-FU) to induce lymphopenia at Day 0. Mice are injected with 150 μg/kg of JL18008-2 or rhIL-7 at Day 2, 4, and 6. Saline is injected as a negative control. Blood samples are obtained by retro-orbital bleeding, without centrifugation, at Day 0, 3, 7, 11, 14, and 21 and analyzed by flow cytometry. As illustrated in FIGS. 3A-3E, at Day 14 and 21, white blood cell (WBC) and lymphocyte (Lym) numbers are increased in the JL18008-2 treated mice as compared to those treated with rhIL-7 or the saline control. At Day 14, CD3+CD4⁺, CD3+CD8⁺, and CD3⁺ T cell populations are significantly elevated in the JL18008-2 treated mice as compared to those treated with rhIL-7 or the saline control. Compared to the negative control, rhIL-7 did not show any effect of improved lymphocyte recovery, possibly due to its short in vivo half-life. These data demonstrate administration of JL18008-2 promotes recovery of white blood cells, lymphocytes, and T cell populations in lymphopenic mice, and suggests JL18008-2 can promote recovery of immune cell populations in lymphopenic subjects.

### Example 5: Bioactivity of JL18008-2 In Vitro

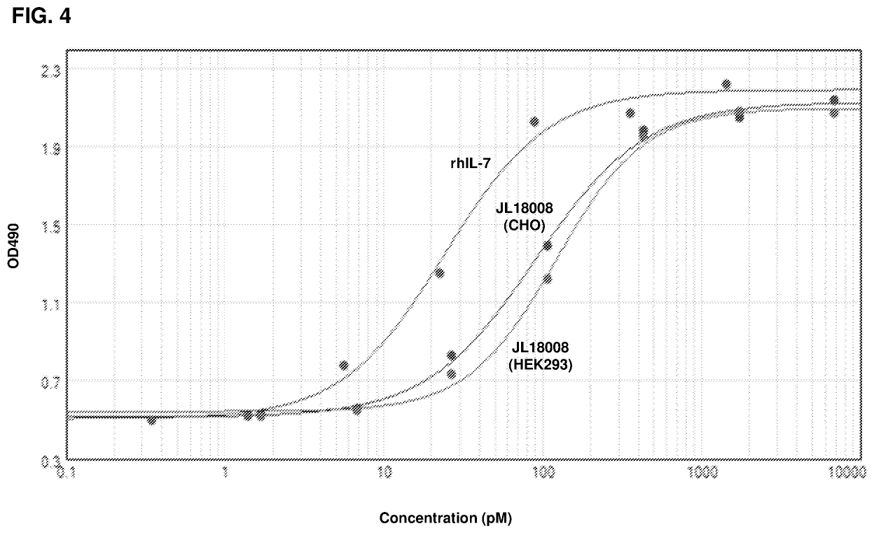

To investigate the bioactivity of JL18008-2 (HSA-IL-7), 2E8 cells (ATCC TIB-239), which are immature, murine B lymphocytes, were seeded into a 96-well plate (5×10⁴ cells/100 μL/well) and JL18008-2 isolated from CHO cells, JL18008-2 isolated from HEK293 cells, or rhIL-7 (Peprotech) were stepwise diluted and added to the wells (100 μL/well). The cells were incubated at 37° C., 5% CO2 for 65 hours, treated with MTS at a concentration of 20 μL/well, and incubated 37° C., 5% CO2 for 6 hours. The resulting absorbance was measured at 490 nm (FIG. 4). EC50 values of cell proliferation were obtained from plotting a dose response curve according to standard techniques. As illustrated in Table 6, the EC50 values of JL18008-2 were 4- to 6-fold higher than the EC50 value of rhIL-7. The lower in vitro activity of the IL-7 fusion proteins in FIG. 4 was likely due to steric hindrance.

TABLE 6
EC50 values of JL18008-2 and rhIL-7

| Sample | EC50 (pM) |
|---|---|
| JL18008-2 (CHO) | 89.7 |
| JL18008-2 (HEK293) | 121.1 |
| rhIL-7 | 24.1 |

We claim:

1. A fusion protein comprising:
a) an IL-7 protein;
b) a hinge region; and
c) an albumin protein,
wherein the hinge region covalently links the IL-7 protein and the albumin protein.

2. The fusion protein of claim 1, wherein the IL-7 protein is human IL-7.

3. The fusion protein of claim 1 or 2, wherein the albumin protein is human serum albumin.

4. The fusion protein of any one of claims 1-3, wherein the hinge region is derived from a sequence comprising the human IgD hinge region.

5. The fusion protein of any one of claims 1-4, further comprising a signal peptide.

6. The fusion protein of claim 5, wherein the signal peptide is derived from a mouse IgG signal peptide sequence.

7. The fusion protein of any one of claims 1-6, wherein the IL-7 comprises SEQ ID NO: 15.

8. The fusion protein of any one of claims 1-7, wherein the albumin comprises SEQ ID NO: 16.

9. The fusion protein of any one of claims 1-8, wherein the hinge region comprises SEQ ID NO: 18.

10. The fusion protein of any one of claims 5-9, wherein the signal peptide comprises SEQ ID NO: 17.

11. The fusion protein of any one of claims 1-9, wherein the fusion protein comprises SEQ ID NO: 19.

12. The fusion protein of any one of claims 1-9, wherein the fusion protein comprises SEQ ID NO: 20.

13. The fusion protein of any one of claims 1-10, wherein the fusion protein comprises in the direction of the N- to C-terminus the albumin protein followed by the IL-7 protein.

14. The fusion protein of any one of claims 1-10, wherein the fusion protein comprises in the direction of the N- to C-terminus the IL-7 protein followed by the albumin protein.

15. The fusion protein of any one of claims 1-14, wherein the serum half-life of the IL-7 is extended in the fusion protein relative to recombinant or exogenous human IL-7 when administered to a subject.

16. The fusion protein of claim 15, wherein the serum half-life of the IL-7 is extended in the fusion protein relative to recombinant or exogenous human IL-7 by at least two hours when administered to a subject.

17. The fusion protein of claim 15, wherein the serum half-life of the IL-7 is extended in the fusion protein relative to recombinant or exogenous human IL-7 by at least four hours when administered to a subject.

18. The fusion protein of claim 16 or 17, wherein when the subject is administered the fusion protein in a range of 50-150 μg/kg, the serum half-life of the human IL-7 is about 195-325 minutes.

19. The fusion protein of claim 18, wherein the serum half-life of the human IL-7 is about 195-225 minutes.

20. The fusion protein of any one of the preceding claims, wherein the fusion protein increases white blood cell counts when administered to a subject.

21. The fusion protein of any one of the preceding claims, wherein the fusion protein increases lymphocyte counts when administered to a subject.

22. The fusion protein of any one of the preceding claims, wherein the fusion protein increases a T cell subpopulation when administered to a subject.

23. The fusion protein of claim 22, wherein the T cell subpopulation is selected from the group consisting of CD3⁺CD4⁺ T cells, CD3⁺CD4⁺ T cells, and CD3⁺ T cells.

24. The fusion protein of any of claims 15-24, wherein the subject has lymphopenia.

25. A composition comprising the fusion protein of any one of claims 1-24 and a pharmaceutically acceptable carrier, diluent, or excipient.

26. A kit comprising the fusion protein of any one of claims 1-24 and a pharmaceutically acceptable carrier, diluent, or excipient.

27. A composition for treating a subject with lymphopenia, the composition comprising:
a) a fusion protein comprising a human IL-7 protein, a hinge region, and human serum albumin protein; and
b) a pharmaceutically acceptable carrier, diluent, or excipient.

28. The composition of claim 27, wherein the hinge region is derived from a sequence comprising the human IgD hinge region.

29. The composition of any of claim 27 or 28, wherein the fusion protein further comprises a signal peptide, wherein the signal peptide is derived from a mouse IgG signal peptide sequence.

30. The composition of any of claims 27-29, wherein the human IL-7 comprises SEQ ID NO: 15.

31. The composition of any one of claims 27-30, wherein the human serum albumin comprises SEQ ID NO: 16.

32. The composition of any one of claims 27-31, wherein the hinge region comprises SEQ ID NO: 18.

33. The composition of any one of claims 29-32, wherein the signal peptide comprises SEQ ID NO: 17.

34. The composition of claim 27, wherein the fusion protein comprises SEQ ID NO: 19.

35. The composition of claim 27, wherein the fusion protein comprises SEQ ID NO: 20.

36. The composition of any one of claims 29-33, wherein the signal peptide is attached to the human IL-7.

37. The composition of any one of claims 29-33, wherein the signal peptide is attached to the human serum albumin.

38. A method for treating lymphopenia, comprising administering to a subject in need of treatment a composition comprising a fusion protein comprising human IL-7, a hinge region, and human serum albumin.

39. The method of claim 38, wherein the fusion protein further comprises a signal peptide, wherein the signal peptide is derived from a mouse IgG signal peptide sequence.

40. The method of claim 38, wherein the fusion protein comprises SEQ ID NO: 1.

41. The method of claim 38, wherein the fusion protein comprises SEQ ID NO: 2.

42. A method for stimulating white blood cell proliferation, lymphocyte proliferation, and/or lymphocyte differentiation, comprising administering to a subject in need of stimulation a composition comprising a fusion protein comprising human IL-7, a hinge region, and human serum albumin.

43. The method of claim 42, wherein the fusion protein further comprises a signal peptide, wherein the signal peptide is derived from a mouse IgG signal peptide sequence.

44. The method of claim 42, wherein the fusion protein comprises SEQ ID NO: 19.

45. The method of claim 42, wherein the fusion protein comprises SEQ ID NO: 20.

46. The method of any one of claims 42-45, wherein the subject has undergone stem cell transplantation.

47. A method for treating immunodeficiency, comprising administering to a subject in need of treatment a composition comprising a fusion protein comprising human IL-7, a hinge region, and human serum albumin.

48. The method of claim 47, wherein the fusion protein further comprises a signal peptide, wherein the signal peptide is derived from a mouse IgG signal peptide sequence.

49. The method of claim 47, wherein the fusion protein comprises SEQ ID NO: 19.

50. The method of claim 47, wherein the fusion protein comprises SEQ ID NO: 20.

51. The method of any one of claims 47-50, wherein the subject has a disease or disorder selected from the group consisting of cancer, human immunodeficiency virus, hepatitis B, hepatitis C, lymphopenia, and sepsis.

52. The method of any one of claims 47-50, wherein the subject has undergone stem cell transplantation.

53. The fusion protein of any one of claims 1-24, wherein the fusion protein binds IL-7 receptor.
